(19) United States
(12) Patent Application Publication
(10) Pub. No.: US 2012/0130307 A1
(43) Pub. Date: May 24, 2012

(54) DEVICE AND METHOD FOR CREATING AN ACCESS TO A HOLLOW ORGAN

(76) Inventor: Walter Pobitschka, Bad Homburg (DE)

(21) Appl. No.: 13/062,165

(22) PCT Filed: Aug. 26, 2009
(86) PCT No.: PCT/DE2009/001190

§ 371 Date: May 16, 2011

(30) Foreign Application Priority Data
Sep. 4, 2008 (DE) 10 2008 045 692.6

(51) Int. Cl. A61M 25/09; A61M 5/142; A61M 25/01

(52) U.S. Cl. 604/151; 604/528

(57) ABSTRACT

The invention relates to a device for creating an access to a hollow organ, comprising a cannula (2), a guide wire (3), and a catheter (4), and it further relates to a special tool for removing the syringe from the catheter after catheterization, and finally it relates to a method for creating an access to a hollow body, wherein the hollow body is punctured by means of a cannula of a syringe, wherein the cannula and the syringe are separated after puncturing, wherein a guide wire is inserted through the cannula into the hollow body, wherein a catheter is inserted along the guide wire into the vessel, and wherein the guide wire is removed after the catheter is placed. The risk of contamination in placing the catheter is to be reduced. This is achieved in that the cannula (2) is part of a syringe (5) having a passage opening (6) for the guide wire (3) and the catheter (4), and that the syringe (5), the catheter (4), and the guide wire (3) are disposed inside a closed system in the form of at least one cladding (7, 8, 9), wherein the device can be actuated from the outside, and the cladding (7) can be perforated by the cannula (2).

The invention relates to a device for creating an access to a hollow organ, comprising a cannula, a guide wire, and a catheter.

The invention further relates to a method for creating an access to a hollow organ, wherein the hollow body is punctured by means of a cannula of a syringe, wherein the cannula and the syringe are separated after puncturing, wherein a guide wire is inserted through the cannula into the hollow body, wherein a catheter is inserted along the guide wire into the vessel, and wherein the guide wire is removed after the catheter is placed.

Finally, the invention relates to a special tool that removes the syringe used according to the device from the catheter.

Catheters are small tubes or hoses of various diameters made of plastic, latex, silicone, metal, or glass that can be used to probe, empty, fill, or flush hollow organs such as the vessels or the bladder. The present invention focuses in particular on creating a permanent access to a blood vessel but includes any and all areas of catheterization.

A permanent access is needed for administering permanent infusions and repeated injections into the venous system of an organism. If a permanent access in created in clinical practice, the vessel is first punctured using the cannula of a syringe that is half filled with saline solution, then the cannula and syringe are separated. Through the cannula, a guide wire is advanced into the vessel up to a predefined position of the catheter outlet, and a catheter is introduced into the vessel along this guide wire. Before placing the catheter, the cannula is threaded out and the access into the vessel may be dilated by pushing a dilator along the guide wire into the access region and dilating it. The dilator is removed after the dilation, and the catheter is introduced into the vessel. The guide wire is removed after the catheter has been placed. The guide wire is held in a person's hand for the entire time—first in front of the catheter, then behind the catheter outlet.

The individual devices—syringe, guide wire, cannula, dilator, catheter—are typically handled in a treatment room in a non-sterile atmosphere. There is a risk of infection. The concentration of bacteria and viruses is high in treatment rooms where patients enter and exit, which can become a danger for weakened organisms.

Based on prior art known from clinical practice, the problem to be addressed by the invention is to provide a device and method for creating an access to a hollow organ wherein the risk of contamination is reduced. In addition, a special tool is to be provided that is used to remove the syringe from the catheter after the access has been created.

The above problem is solved with respect to the device by the features of patent claim 1. According to this claim, a device of the type discussed is designed such that the cannula is an integral part of a syringe with a passage opening for the guide wire and the catheter and in that the syringe, the catheter, and the guide wire are arranged inside an enclosed system in the form of at least one cladding such that the device can be operated from outside and the cannula can perforate the cladding.

The above problem is solved with respect to the special tool by the features of patent claim 23. According to this claim, the special tool for separating the syringe of the device according to the invention from the catheter after creating the access to the hollow organ is equipped with two jaws that are connected by a hinge for grasping the syringe and with crushing means on the inside of the jaws, wherein the jaws are a component of a pliers and can be moved towards and away from one another via a plier lever each.

The above problem is solved with respect to the method by the features of patent claim 28. According to this claim, the method of the type discussed is designed such that the guide wire reaches the cannula through a passage opening of the syringe and in that the syringe, the catheter and the guide wire are contained in an enclosed system, in particular in at least one cladding, wherein they are operated from outside and the cannula perforates the cladding before it punctures the hollow organ.

Based on the prior art known from clinical practice, it has been found that creating an access to a hollow organ poses serious contamination risks. It has been found according to the invention that the contamination risk can be reduced if the required components are arranged in an enclosed system. The enclosed system comprises at least one cladding which however can be perforated by the cannula. Contamination with pathogens from the environment is excluded by this shielding of the components from the environment as long as the system is closed. The system is only opened when the cannula penetrates into the organism, wherein the cannula just punctures the cladding. The time during which the cannula is in contact with ambient air thus is very short. It is obvious that the contamination risk is reduced according to the invention. It should be pointed out with respect to the special tool as a supplement to the device that the syringe in it has no more function after catheterization and can be removed as ballast.

The passage opening for guide wire and catheter could run through the plunger of the syringe and correspond with the cannula-side opening of the tube of the syringe so that these components can get to the desired position in the hollow body or blood vessel.

Since the device has several components, these could be located in separate claddings that can be connected to one another due to the transfer of device components to the application site. In particular, there are the cladding of the syringe, the cladding of the insertion area for the guide wire and the catheter that can be separated from, or connected to, one another. The site where the cannula cladding is opened should be closable so that the components of the device will not be contaminated by entering outside air. A separate cladding could also be provided where the syringe is opened or closed, in particular for the holding means of the cannula and the closing means at the end of the syringe plunger. The separate cladding allows the holding means to be turned without influencing the claddings of the syringe or of the cannula. In an embodiment wherein a cladding contains both the syringe and the holding means, the cladding could be unnecessarily strained in the area intended for the syringe. Also, a separate cladding of the holding means leaves more options for the design of the syringe cladding that has to be very tight in the tube section.

The claddings may consist of a soft and non-tear plastic that is transparent or at least semi-transparent and facilitates handling of the component of the device from outside. The cladding should consist of a readily penetrable material such as latex in the perforation section of the cannula.

In addition, a sterile patch could be provided on the syringe that is used to cover the puncture site. The sterile patch also helps to minimize the exposure of the puncture site to the atmosphere. The sterile patch can be applied immediately after the puncture or after the catheter has been placed.

Before the device according to the invention is used, the cannula and its cladding could in addition be housed in a removable protective sleeve. In this way, the sensitive front end of the readily perforable cladding is protected from mechanical impact and loss of cladding.

The cladding of the cannula could be designed for being pushed back after perforation so that the cannula is accessible and can be handled as usual.

The syringe may comprise a dilator for widening the entrance into the vessel. It is useful if the dilator is detachably connected to the tube of the syringe. For example, an add-on with a female thread into which the male thread of the dilator could be screwed could be provided on the tube. Dilation of the hollow body is required so that the catheter can be inserted. The dilation can either be performed in that the dilator is slid over the cannula while the latter is still inside the puncture hollow body or after the cannula has been disposed of by sliding the dilator along the guide wire.

Furthermore, a holding means for the cannula may be provided on the tube of the syringe that, in addition to its holding function, also fulfills a closing and opening function. It could be set to various inside widths, first, to hold the cannula tight and firm according to its cross section, and second to form the passage opening for the device components to be threaded through, such as guide wire and catheter. The holding means could also comprise a separate cladding. The cladding of the syringe could adjoin this cladding. With respect to the movement of the plunger in the tube, its cladding could at least partially shaped as a bellows.

A closing means could be provided at the end of the plunger of the syringe that opens or closes the passage opening and that can preferably be set to the various cross sections of the guide wire and catheter. The design solution could be a tightly closing cover that more or less opens the inlet or outlet of the closing means when turned open or shut depending on the turn position. The desired cover setting should of course be lockable. As an alternative to integrating the closing means into the syringe cladding, a separate cladding could be provided for the closing means—like the one for the holding means—to separate the turning process when opening or closing the cover using a knurled ring.

A cladding could be provided for the section of the device adjacent to the syringe that contains the guide wire and the catheter. Since the priority in the handling zone where the guide wire is threaded into the passage opening is easy operation, the cladding could generally be designed as a bellows.

Volume changes of the medium contained can occur wherever the claddings remain closed, namely in the region of the syringe plunger and where the guide wire and catheter are inserted locally before the syringe. The enclosed system, except for the perforation site, can be maintained if the pressure differences are equalized within the system. A pressure equalization facility could be provided that is in sterile fluid communication with at least one system. In other words: Displaced air is advantageously not released into the ambient air but captured in a pressure equalization facility and will be available there to prevent a vacuum from occurring in other processes. The device according to the invention can be used on a patient on site and be designed a single-use item. Standardized single-use items according to the invention are an enormous benefit when working under conditions of greatly reduced contamination.

The guide wire and catheter could be housed in a storage container arranged behind the cladding for the guide wire and catheter so that the introduction of guide wire and catheter can also take place within the enclosed system. This storage container is preferably coupled to the device in a sterile manner and ensures the connection to the cladding in which the device components are handled. When the device is removed and disassembled, the storage container may change its function and become a container for disposal. It is a particular advantage for easy handling if the catheter and wire are preassembled in the storage container so that the catheter is already pulled over the wire.

A manifold for various ports could be provided between the cladding for the guide wire and catheter and the storage and disposal container. These ports could be used for supplying drugs or normal saline solution to maintain a fluid column inside the catheter. It is essential that the ports are arranged tightly sealed on the manifold so that the enclosed system is not compromised. The ports could, for example, be welded on.

A special advantage is the assignment of specially sheathed valves to each port. These prevent back flow of fluids that may escape from the hollow organ which could provide a culture medium for microorganisms. Back flow can only occur to the valve or stop cock, the section of the catheter to be cared for is reduced or simplified. Sheathed valves also pose an advantage in reverse direction if the inlet is cleaned with alcohol. The cleaning agent or contaminated air cannot enter the catheter in this way. It is also possible to administer a dosed quantity of a drug.

The cladding could comprise a sterile connection for a pumping system. This is advantageous to generate a slight positive pressure by sterile air inside the cladding, which results in a directed airflow. A pumping system can especially be used when the cannula is removed and moved towards the storage and disposal container. The holding means is opened, and contaminated air could undesirably enter into the device, especially into the catheter. The sterile air pumped in virtually builds up a pressure wall that prevents the inflow of contaminated air. This process takes a short time only. The system can be depressurized using the pressure equalization facility. This additional effort is indicated for patients that are at a particular risk of contamination.

After the guide wire has been inserted into the hollow body, the cannula has to be removed from the organism. The plunger of the syringe is in a depressed position at the tube outlet since it has been actuated when puncturing the hollow organ, particularly the vessel from outside through the cladding. The catheter is advanced into the tube and towards its outlet in the vicinity of the holding means. For multiple-lumen catheters, a central lumen has to be positioned directly in the center of the passage opening of the plunger. The holding means for the cannula is opened so that the cannula can be moved through the catheter to the emptied storage and disposal container.

According to a particularly preferred embodiment of the invention, the cannula could be magnetized and interact with a magnetic means. After the holding means is opened, the cannula is moved contactless through the catheter to the storage and disposal container, which only fulfills its disposal function now. The magnetic means could be designed as a ring magnet or a magnet that can be swung open. The strength of the magnetic field could be ensured by a permanent magnet. A magnetic means that utilizes electrical magnetic fields is another option.

When the cannula is disposed of, the catheter is positioned at the desired site through the open holding means along the guide wire. Then the guide wire is withdrawn via the bellows of the cladding for guide wire and catheter and placed in the storage and disposal container that then fulfills its disposal function only and can be separated in a sterile manner.

Then the entry point of the catheter into the organism is treated in a sterile manner. This requires removal of the sterile patch to reuse it after proper disinfection for covering the site. Alternatively, the sterile patch can be used for the first time now to temporarily affix the catheter until the latter is finally affixed in the conventional way. According to yet another alternative embodiment, at least two sterile patches can be provided so that the first patch is placed on the skin immediately after puncturing, for example, a blood vessel, is removed for catheterization, and the second patch is placed on the catheter entry point after the guide wire has been removed.

After the catheter entry point has been sterilized, the syringe with tube, plunger, and dilator is still on the catheter. This is uncomfortable for the patient. According to an improved embodiment of the invention, a tool is provided according to the invention that can be used to remove the syringe that still sits on the catheter and has now become useless after the access has been created. This special tool could be handled like pliers, and the crushing jaws could preferably have the form of molded parts that are exactly matched to fit the tube and plunger of the syringe and to the holding means for the cannula and the closing means at the end of the plunger. The syringe could consist of an easily destructible material, particularly a plastic that can readily be crushed by metal teeth. The tool could further comprise a collecting means for the fragments of the destroyed syringe. The jaws could have openings through which the fragments of the destroyed syringe get into the collecting means.

Regarding the method, the guide wire is positioned in the blood vessel as usual after the puncture of the hollow organ, especially a blood vessel. It is novel in this arrangement that the guide wire is conducted from the storage and disposal container through the passage opening of the plunger that is depressed down to the tube outlet during the puncture and through the cannula, that the system is operated from the outside while all processes take place inside a system. After the guide wire is positioned, the catheter is advanced, first to the tube, through the passage opening of the syringe. Then the magnetized cannula is removed from a holding means and moved using the magnetic means through the catheter to a storage and disposal container that at this point in time just functions as a disposal container. As explained in connection with the device according to the invention, a pumping system connected to the cladding could introduce sterile air or an inert gas into the cladding, especially when removing the cannula from the holding means and contaminated air from the area between the tube and the hollow organ could ingress into the cladding of the syringe or in the catheter, respectively. The pressure is generally equalized within the system wherever the volume within the cladding changes.

After disposing of the cannula in the storage and disposal container, the catheter is advanced through the opened holding means on the tube along the guide wire into the hollow organ. After the catheter has been placed, the guide wire is pulled out of the hollow organ and placed in the storage and disposal container.

Sterile air can be pumped into the system during this process, however the passage opening of the holding means can be set to the cross section, and the risk of contamination is low due to the small wire cross section.

The storage and disposal container is cut off in a sterile manner by welding after it has received the used cannula and the used guide wire. As described in conjunction with the tool of the invention, the syringe is removed using the special tool to allow the patient to be comfortable. For the same reason, the remnants of the cladding between the syringe and the connection for the storage and disposal container are removed, for example by using scissors. Alternatively, tear-off means could be provided that would allow removal of the cladding material along predetermined tearing points. In the end, only the catheter will remain, optionally with a manifold and ports.

There are various ways of advantageously developing and improving the rationale of this invention. For this purpose, reference is made to the claims that are subordinate to patent claim 1 and to the explanation of an embodiment of the invention with reference to the enclosed figure. Generally preferred designs and improvements of the concept of the invention are described in conjunction with explaining an embodiment of the invention. Wherein:

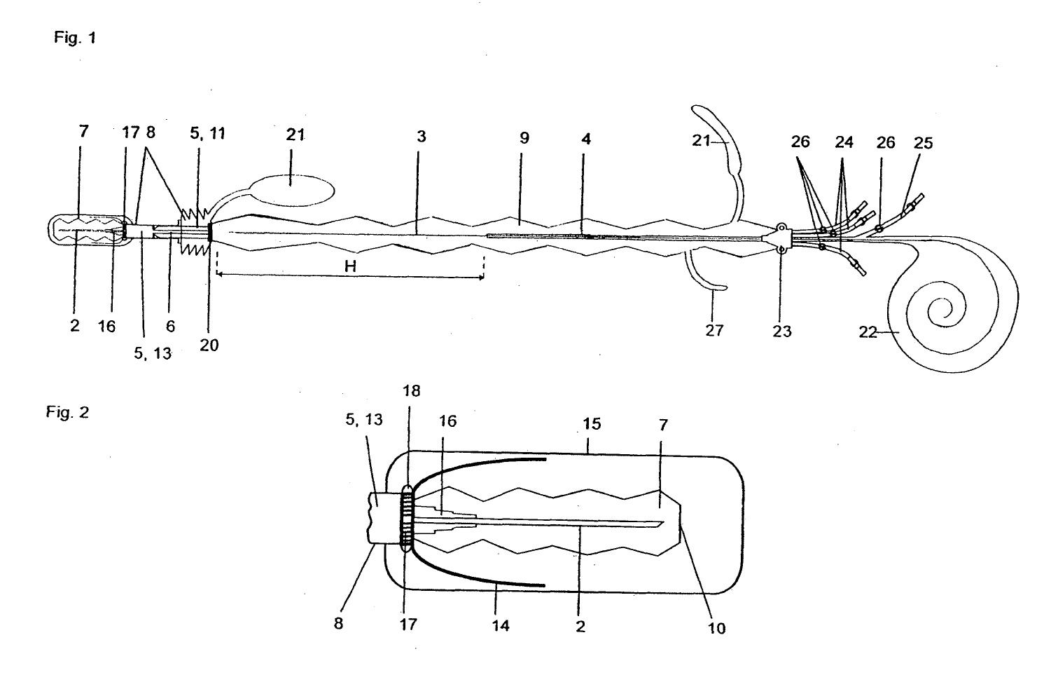

FIG. 1 shows a diagrammatic lateral view of the device according to the invention in idle position;

FIG. 2 shows a diagrammatic enlarged view of a detail from FIG. 1 concerning the protective sleeve;

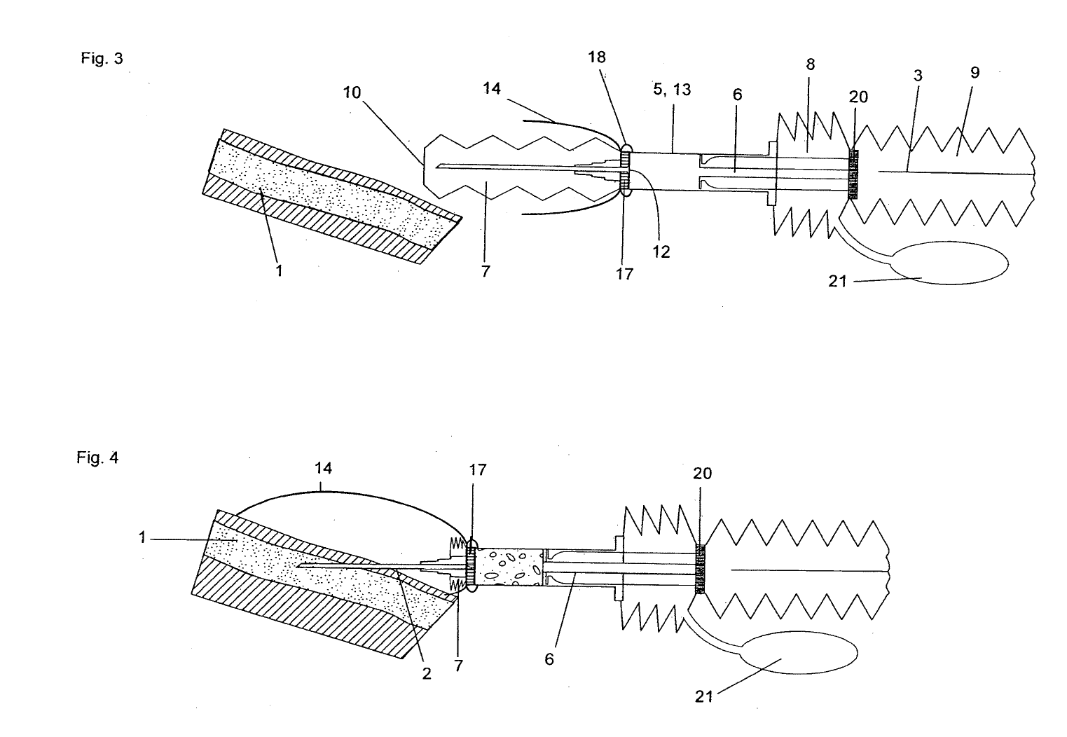

FIG. 3 shows a diagrammatic enlarged partial view of the subject matter from FIG. 1 after removal of the protective sleeve;

FIG. 4 shows a diagrammatic enlarged partial view of the subject matter from FIG. 1 after puncturing the cladding in the perforation area and puncturing the blood vessel;

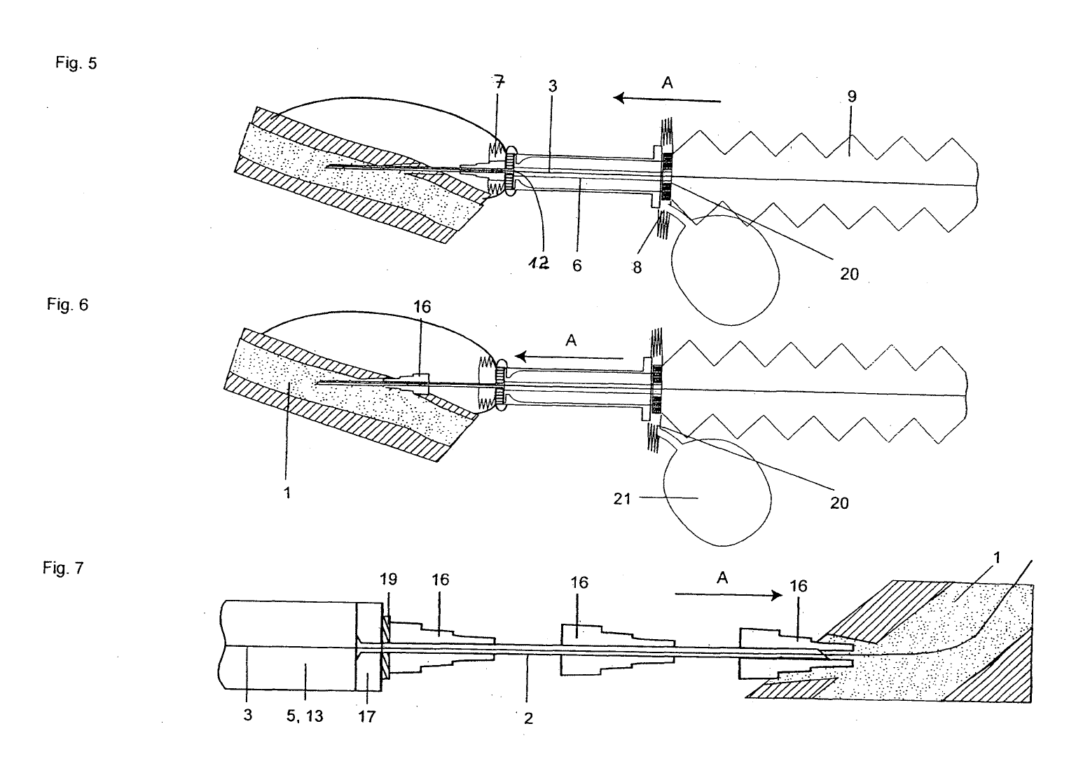

FIG. 5 shows a diagrammatic enlarged partial view of the subject matter from FIG. 1 during the introduction of the guide wire through the syringe and the cannula into the blood vessel;

FIG. 6 shows a diagrammatic enlarged partial view of the subject matter from FIG. 1 during the use of the dilator;

FIG. 7 shows a diagrammatic sketch of how to handle the dilator;

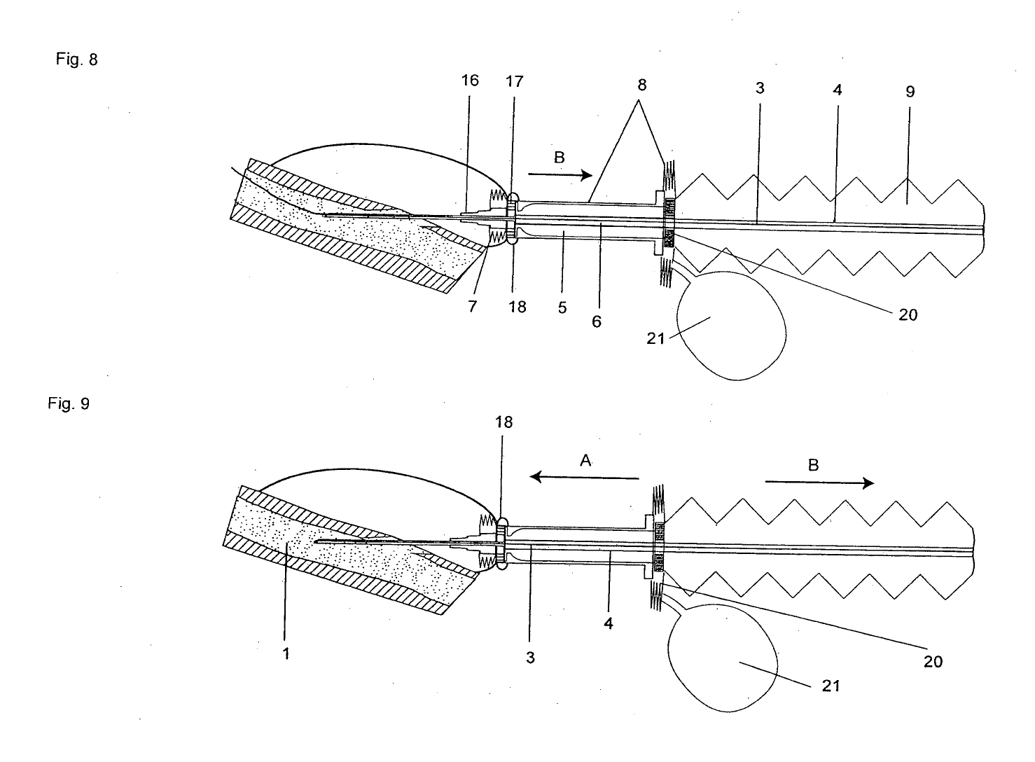

FIG. 8 shows a diagrammatic enlarged partial view of the subject matter from FIG. 1 after the use of the dilator;

FIG. 9 shows a diagrammatic enlarged partial view of the subject matter from FIG. 1 with the catheter conducted through the syringe to the closed holding means on the tube;

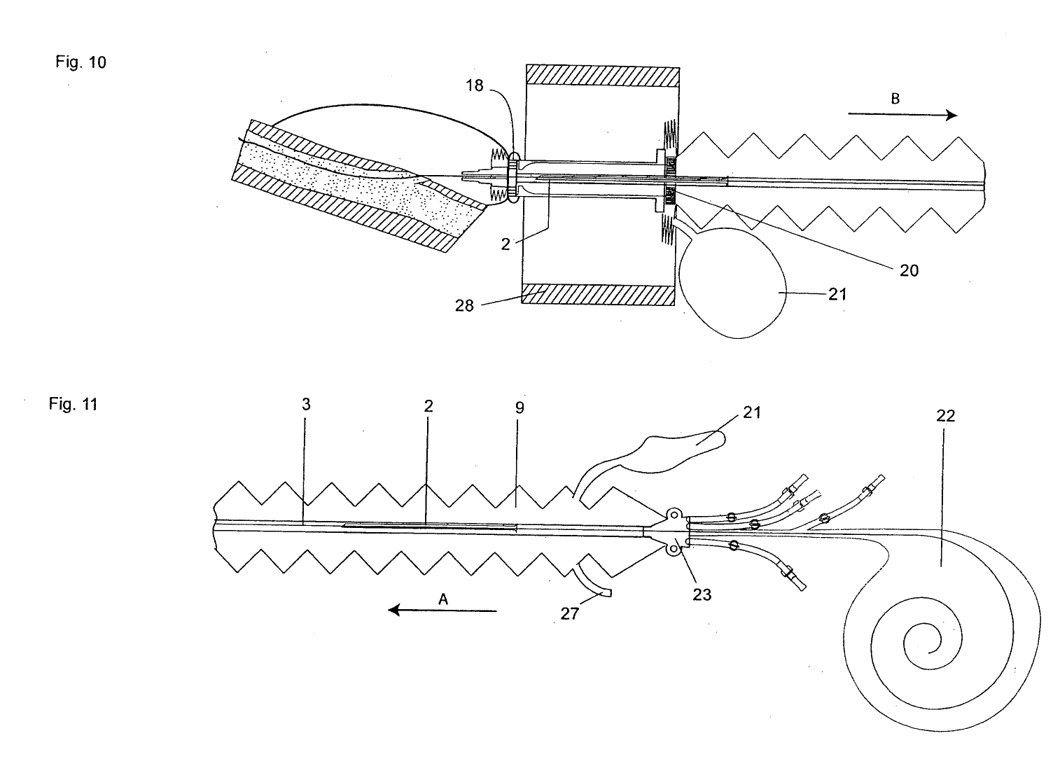

FIG. 10 shows a diagrammatic enlarged partial view of the subject matter from FIG. 1 during the removal of the cannula through the catheter using the magnetic means;

FIG. 11 shows a diagrammatic enlarged partial view of the subject matter from FIGS. 1 and 11 concerning the path of the cannula to the storage and disposal container;

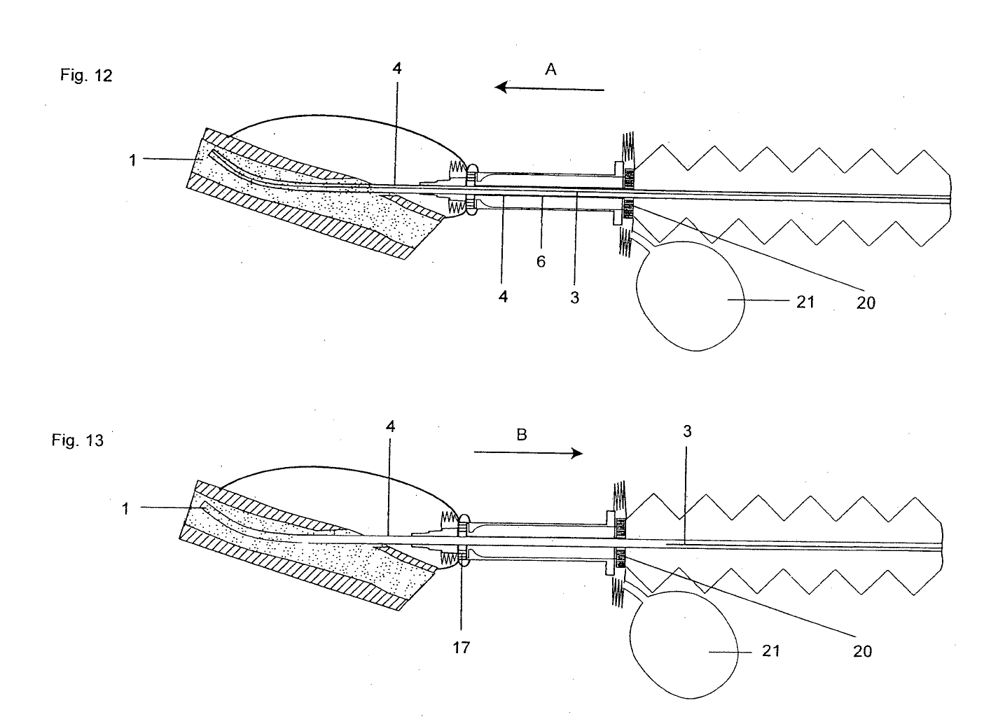

FIG. 12 shows a diagrammatic enlarged partial view of the subject matter from FIG. 1 during the introduction of the catheter through the syringe into the prepared blood vessel;

FIG. 13 shows a diagrammatic enlarged partial view of the subject matter from FIG. 1 during the removal of the guide wire from the blood vessel after the catheter has been introduced;

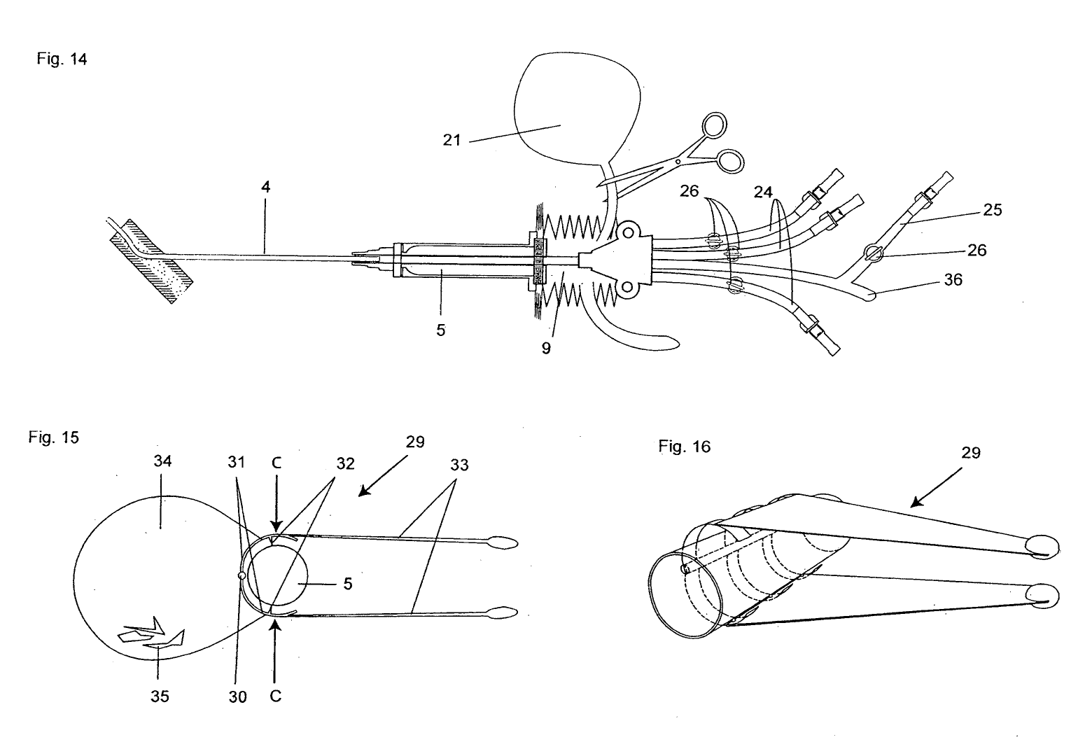

FIG. 14 shows a diagrammatic enlarged partial view of the subject matter from FIG. 1 after the access has been created;

FIG. 15 shows a diagrammatic sketch of the special tool according to the invention for separating the syringe from the catheter; and

FIG. 16 shows a diagrammatic partial perspective view of the subject matter from FIG. 15.

FIGS. 1 to 14 present a device for creating an access to a hollow organ in the form of a blood vessel 1, said device having a cannula 2, a guide wire 3, and a catheter 4. The cannula 2 is an integral part of a syringe 5.

According to the invention, the syringe 5 is equipped with a passage opening 6 for the guide wire 3 and the catheter 4. The syringe 5, the catheter 4 and the guide wire 3 are arranged in an enclosed system according to FIG. 1. The enclosed system comprises multiple airtight transparent and non-tear claddings 7, 8, 9 made of plastic through which the syringe 5, the guide wire 3, the cannula 2, and the catheter 4 can be actuated from outside. In the present embodiment, the cladding 7 can be perforated by the cannula 2 in the perforation zone 10. The cladding 7 consists of latex in the perforation zone 10.

The passage opening 6 runs through the plunger 11 of the syringe 5 and, when the plunger is depressed, see FIGS. 4 to 6, 8 to 10, communicates with the cannula side passage opening 12 of the tube 13 of the syringe 5.

A sterile patch 14 that is used to cover the puncture site is provided on the syringe 5. FIG. 2 shows that the cannula 2 and the cladding 7 there are arranged inside a protective sleeve 15 when in idle position. The cladding 7 in the region of the cannula 2 has the form of a bellows that can be retracted towards the tube 13 after perforation.

The syringe 5 further comprises a dilator 16 that is arranged on the holding means 17 for the cannula 2. The holding means 17 is associated with the tube 13 of the syringe 5 and enclosed in its own cladding 18. The holding means 17 comprises a knurled ring that is actuated from outside the cladding 18, wherein a cover not shown here can be set to various cross sections, i.e. the cross sections of the cannula 2, the guide wire 3, and the catheter 4. The separate cladding 18 was selected here to make turns of the knurled ring of the holding means 17 ineffective with respect to the cladding 8 of the syringe 5.

FIGS. 6 to 8 show that the dilator 16 is detachably attached to the holding means 17. FIG. 7 includes a threaded component designated 19 into which the dilator 16 is screwed. The dilator 16 is detached from the threaded 19 when it is used at the puncture site. After dilating the vessel entrance, the dilator 16 is once again attached to the threaded component 19.

The cladding 8 of the syringe 5 has the form of a bellows in the area of the plunger 11 while it sits smoothly against the tube wall in the area of the tube 13 and facilitates as normal an operation of the syringe 5 as possible.

The claddings 8 and 9 can be connected, which is required to perform the procedure. For this purpose, a closing means 20 is provided at the end of the plunger 11 of the syringe 5 that provides or closes access to the passage opening 6 and can be set to the various cross sections of the guide wire 3 and the catheter 4, thus also performing a centering function.

The cladding 9 for the introduction of the guide wire 3 and catheter 4 and for the later withdrawal of the guide wire 3 also has the form of a bellows and is adjoined directly to the syringe 5. The handling zone of the cladding 9 is designated H. This is where the guide wire 3 and the catheter 4 are threaded through the closing means 20 into the passage opening 6 and from there through the holding means 17 on to the blood vessel 1. The claddings 8 and 9 each comprise a pressure equalization facility 21.

A storage and disposal container 22 from which the guide wire 3 and the catheter 4 can be conducted to the syringe 5 and eventually to the blood vessel 1, is arranged behind the cladding 9. In the present embodiment, the catheter 4 and the guide 3 are preassembled, i.e. the catheter 4 is already pulled over the guide wire 3 inside the storage and disposal container 22.

A manifold 23 for various ports 24, 25 for the catheter 4 is provided between the cladding 9 and the storage and disposal container 22. The ports 24, 25 may, for example, be connected to infusion sets or other coupling units and each include a valve 26 with a sterile sheath. The port 25 is only activated after the storage and disposal container 22 has been disconnected from the system. Port 25 is used to feed into the central lumen of the catheter 4 that has four lumina here. The ports 24 supply the three other lumina of the catheter 4. The storage and disposal container represents, as it were, an extension of the central lumen of catheter 4.

The cladding 9 also comprises a port 27 for sterile connection of a pumping system not shown here with which sterile air is introduced to the cladding 9 and optionally cladding 8 as well to build a sterile front against contaminated air from outside that could enter when the holding means 17 is opened, especially when the cannula 2 is moved towards the storage and disposal container 22. The cannula 2 is magnetized when the guide wire 2 is positioned in the blood vessel 1, the magnetized cannula 2 is moved using a magnetic means 28 and transported through the open holding means 17 and the catheter 4 in the direction of arrow B to the storage and disposal container 22. The magnetic means 28 is designed as an annular permanent magnet here. FIGS. 10 and 11 show the path of the cannula 2 to the storage and disposal container 22.

When the cannula 2 has been disposed of and the guide wire 3 has been retracted and the access of the catheter 4 to the blood vessel 1 has been created, the syringe 5 is mere ballast and no longer needed. The syringe 5 is separated from the device using the special tool 29. The tool 29 comprises two jaws 31 connected by a hinge 30 for grasping the syringe 5. Crushing means 32 pointing inwards are provided on the inner sides of the jaws 31. The jaws 31 are the components of a pliers and can be moved towards one another via one plier lever 33 each as indicated by arrows C, in which process the syringe 5 is destroyed, or they can be moved away from one another in opposite direction of the arrows C.

The jaws 31 are molded parts that are exactly matched to fit the tube 13 with holding means 17 and the plunger 11 with closing means 20 of the syringe 5. All the components of the syringe 5 consist of a readily destructible material, in the embodiment shown here of plastic.

It is apparent from FIG. 15 that the tool 29 comprises collecting means 34 for the fragments 35 of the syringe 5 to be destroyed. The destruction process is to free the catheter 4 from the syringe 5. The cladding 9 with the pressure equalization means 21 is removed as well—here as shown in FIG. 14 using an unspecified scissors.

The following steps are performed to create an access to the blood vessel (and can well be traced using FIGS. 3 through 15).

The device according to the invention is still in idle position in FIG. 3. The cladding 7 of the cannula 2 is still intact. The cannula 2 rests firmly and tightly sealed in the holding means 17.

FIG. 4 shows that the cladding 7 has been perforated and retracted to the holding means 17, the blood vessel 1 was punctured and the sterile patch 14 was placed over the puncture site. The normal saline solution that is contained in the tube 13 of the syringe 5 is injected into the blood vessel 1 by actuating the plunger 11, wherein the bellows of cladding 8 is contracted and the sterile air from cladding 8 is displaced into the pressure equalization facility 21.

FIG. 5 shows that the plunger 11 with the passage opening 6 connects directly to the passage opening 12 of the holding means 17 for cannula 2. There is a connection between the passage opening 6 of the plunger 11 and cannula 2 that sits firmly and tightly in the holding means 17 so that no contamination can take place. The closing means 20 located at the end of the plunger 11 is closed during this injection process and opened afterwards to transport the guide wire 3 through the passage opening 6 and the cannula 2 into the blood vessel 1. Advancement is brought about via the handling zone H in the direction of the arrow A.

FIGS. 6 to 8 show the dilation of the entrance to the blood vessel 1 using the dilator 16. The dilator 16 comprises a male thread that is not shown here and that interacts with the female thread of the threaded component 19 on the holding means 17. The thread has one turn only and can easily be operated from outside—using the sterile patch 14 and the retracted cladding 7. The dilator 16 is pushed over the cannula 2 in the direction A towards the puncture site and to the blood vessel 1. After dilation, the dilator 16 is pushed back on the cannula 2 in the direction of arrow B and attached to the closed holding means 17. The closing means 20 may remain open during this process since the holding means 17 is closed and the cannula 2 sits firmly and tightly in it so that there can be no contamination from outside.

After the dilation process is completed and the dilator 16 is once again attached to the threaded component 19, the catheter 4 is advanced in the direction of arrow A from the cladding 9 through the open closing means 20 and through the passage opening 6 of the syringe 5 into the tube 13 until it is located directly in front of the closed holding means 17. This is a catheter 4 with four lumina. The central lumen communicates with the cannula 2 and the storage and disposal container 22 so that the former can be removed in a next step through the catheter 4 in the direction of arrow B.

Movement of the cannula 2 in the direction of arrow B is shown in FIGS. 10 and 11. The holding means 17 is slightly opened and the magnetized cannula 2 is moved along the guide wire 3 through the catheter 4 to the storage and disposal container 22 using the magnetic means 28. The storage and disposal container 22 in the embodiment shown represents, as it were, an extension of the central lumen of catheter 4. The holding means 17 has to be open while the cannula is removed so that ambient air can enter. The sterile patch 14 cannot ensure absolute sterility, of course. Therefore, sterile air is pumped via a pumping system not shown here and connected to the cladding 9 via port 27 into the claddings 9 and introduced via the open closing means 20 into the cladding 8 or into the tube 13, respectively. A flow in the direction of arrow A or slight positive pressure is generated to prevent the ingression of contaminated air from the section between the tube 13 or holding means 17 and the blood vessel 1 into the catheter 4. The pressure within the system is equalized via pressure equalization facilities 21.

After disposing of the cannula 2 in the storage and disposal container 22, the catheter 4 is advanced in the direction of the arrow A along the guide wire 3 through the open closing means 20, through the passage opening 6 of the syringe 5, and through the open holding means 17 into the blood vessel 1 until it has reached the desired position, as shown in FIG. 12.

FIG. 13 shows that the guide wire 3 is removed from the blood vessel and moved in the direction of arrow B into the storage and disposal container 22 after the catheter 4 has been positioned in the blood vessel 1.

FIGS. 14 to 16 show follow-up steps after the access has been created. First, the storage and disposal container 22 with the disposed of cannula 2 and the disposed of guide wire 3 is cut off the catheter 4 by welding in a sterile manner, which creates an additional port 36 through which saline solution or drugs can be introduced. The syringe 5 is removed using the tool 29. Remnants of the cladding 9 and the storage and disposal container are also removed.

For information about other features that are not shown in the figures, see the general part of the description.

In conclusion, it should be pointed out that the teaching according to the invention is not limited to the above embodiment.

List of reference signs

| | |
|---|---|
| 1 | Blood vessel |
| 2 | Cannula |
| 3 | Guide wire |
| 4 | Catheter |
| 5 | Syringe |
| 6 | Passage opening |
| 7 | Cladding |
| 8 | Cladding |
| 9 | Cladding |
| 10 | Perforation zone |
| 11 | Plunger |
| 12 | Passage opening |
| 13 | Tube |
| 14 | Sterile patch |
| 15 | Protective sleeve |
| 16 | Dilator |
| 17 | Holding means |
| 18 | Cladding |
| 19 | Threaded component |
| 20 | Closing means |
| 21 | Pressure equalization facility Connecting means |
| 22 | Storage and disposal container |
| 23 | Manifold |
| 24 | Port |
| 25 | Port |
| 26 | Valve |
| 27 | Port |
| 28 | Magnetic means |
| 29 | Tool |
| 30 | Hinge |
| 31 | Jaws |
| 32 | Crushing means |
| 33 | Plier lever |
| 34 | Collecting means |
| 35 | Fragments |
| 36 | Port |
| A | arrow |
| B | arrow |
| C | arrow |
| H | Handling zone |

1.-37. (canceled)

38. A device for creating an access to a hollow organ, said device comprising:
a cannula; a guide wire; and a catheter,
wherein the cannula is a component of a syringe having a passage opening for the guide wire and the catheter and wherein the syringe, the catheter, and the guide wire are arranged inside an enclosed system of at least one cladding, wherein the device can be actuated from outside, and wherein the cladding can be perforated by the cannula.

39. The device according to claim 38, wherein the passage opening runs through a plunger of the syringe and communicates with a cannula-side passage opening of a tube of the syringe.

40. The device according to claim 38, wherein multiple claddings are provided that can be connected to one another.

41. The device according to claim 38, wherein the cladding is made of latex in the perforation zone.

42. The device according to claim 38, wherein a sterile patch that is used to cover the puncture side is provided on the syringe.

43. The device according to claim 41, wherein the cannula and the cladding there are arranged inside a removable protective sleeve when in idle position.

44. The device according to claim 38, wherein the cladding is designed as a bellows in the region of the cannula and can be retracted after perforation.

45. The device according to claim 38, wherein the syringe comprises a dilator.

46. The device according to claim 38, wherein the syringe comprises on its tube a holding means for the cannula that can preferably be set to various cross sections, in particular to the cross section of the catheter.

47. The device according to claim 46, wherein the holding means comprises a separate cladding.

48. The device according to claim 45, wherein the dilator is detachably attached to the holding means.

49. The device according to claim 40, wherein the cladding of the syringe is at least partially designed as a bellows in the region of the plunger.

50. The device according to claim 38, wherein a closing means that opens or closes the passage opening and can preferably be set to the various cross sections of the guide wire and the catheter is provided at the end of the plunger of the syringe.

51. The device according to claim 40, wherein a cladding for the guide wire and the catheter is provided that is preferably designed as a bellows and is adjoined directly to the syringe.

52. The device according to claim 40, wherein the cladding of the syringe, in the region of the plunger, and the cladding for the guide wire and the catheter, each comprise a pressure equalization facility.

53. The device according to claim 38, wherein the cladding for the guide wire and the catheter is adjoined by a storage and disposal container from which the guide wire and the catheter can be conducted to the syringe and eventually to the hollow organ.

54. The device according to claim 53, wherein the catheter is already preassembled with the guide wire.

55. The device according to claim 53, wherein a manifold for various ports is provided between the cladding for the guide wire and the catheter and the storage and disposal container.

56. The device according to claim 55, wherein a valve having a sterile sheathing is associated with the ports that can preferably be used for sterile connections.

57. The device according to claim 38, wherein the cladding comprises a port for connecting a pumping system in a sterile manner.

58. The device according to claim 38, wherein the cannula is magnetized.

59. The device according to claim 58, wherein a magnetic means is provided that interacts with the magnetized cannula that gets the latter moving.

60. A method for creating an access to a hollow body, said method comprising:
puncturing the hollow body is using the cannula of a syringe;
separating the cannula and syringe after the puncture;
inserting a guide wire through the cannula into the hollow body;
inserting a catheter into the vessel along the guide wire and removing the guide wire after the catheterization,
wherein the guide wire is conducted to the cannula through a passage opening of the syringe, wherein the syringe, the catheter and the guide wire are contained in an enclosed system, in particular in at least one cladding, and are actuated from outside, and wherein the cannula perforates the cladding before it punctures the hollow organ.

61. The method according to claim 60, wherein the catheter is advanced, first to the tube, through the passage opening of the syringe.

62. The method according to claim 61, wherein the cannula is removed from a holding means after the guide wire is positioned in the hollow organ and after the catheter is positioned in front of the outlet of the tube, and moved through the catheter to a storage and disposal container.

63. The method according to claim 62, wherein the cannula is magnetized and is moved using a magnetic means.

64. The method according to claim 60, wherein a pumping system connected to the cladding could introduce sterile air or an inert gas into the cladding, especially when removing the cannula from the holding means and contaminated air from the area between the tube and the hollow organ could ingress into the catheter.

65. The method according to claim 60, wherein a pressure equalization process takes place at those sites inside the system where the volume within the cladding changes.

66. The method according to claim 61, wherein, after the cannula has been disposed of in the storage and disposal container, the catheter is advanced along the guide wire into the hollow organ and in that the guide wire is removed from the hollow organ after catheterization and placed in the storage and disposal container.

67. The method according to claim 66, wherein the storage and disposal container is cut off by welding in a sterile manner after having received the cannula and the guide wire.

68. The method according to claim 66, wherein the syringe is removed using the tool.

69. The method according to claim 66, wherein remnants of the cladding between the syringe and the storage and disposal container are removed.
